(19) United States
(12) Patent Application Publication
(10) Pub. No.: US 2024/0189006 A1
(43) Pub. Date: Jun. 13, 2024

(54) DEVICE FOR MIXING BONE CEMENT

(71) Applicant: Heraeus Medical GmbH, Wehrheim (DE)

(72) Inventors: Sebastian VOGT, Wehrheim (DE); Thomas KLUGE, Wehrheim (DE)

(21) Appl. No.: 18/531,844

(22) Filed: Dec. 7, 2023

(30) Foreign Application Priority Data
Dec. 13, 2022 (EP) 22213010.6

(51) Int. Cl. A61B 17/88; B01F 23/50; B01F 33/501; B01F 35/71; B01F 35/75

(52) U.S. Cl. CPC A61B 17/8833; B01F 23/565; B01F 33/5011; B01F 35/7131; B01F 35/7163; B01F 35/7546; A61B 2017/00477; A61B 2017/8838

(57) ABSTRACT

A device for mixing bone cement comprises a first hollow body which encloses a first cavity for receiving a powder and has a first opening, and a second hollow body which encloses a second cavity for receiving an ampoule and has a second opening. The first hollow body and the second hollow body are connectable to one another in such a way that the first opening and the second opening are fluidically connected to one another. The first hollow body is elastic so that a content of the first hollow body can be mixed from the outside by kneading.

### CROSS-REFERENCE TO RELATED DOCUMENTATION

This application claims priority pursuant to 35 U.S.C. 119(a) to European Application No. 22213010.6, filed Dec. 13, 2022, which application is incorporated herein by reference in its entirety.

### FIELD

The invention relates to a device for storing and/or mixing bone cement.

### BACKGROUND

Bone cement is usually produced by mixing a powder with a liquid. For example, polymethyl methacrylate (PMMA) bone cements are known which are composed of a liquid monomer component and a powder component. The monomer component generally contains the monomer methyl methacrylate and in particular an activator dissolved therein, such as N,N-dimethyl-p-toluidine. The powder component, also referred to as bone cement powder, has one or more polymers which are produced on the basis of methyl methacrylate and comonomers such as styrene, methyl acrylate or similar monomers by polymerization, preferably suspension polymerization, and in particular a radiopaquer and/or the initiator dibenzoyl peroxide. When the powder component is mixed with the monomer component, a plastically deformable dough, the actual bone cement, is produced, for example by the polymers of the powder component swelling in the methyl methacrylate. When the powder component is mixed with the monomer component, the activator N,N-dimethyl-p-toluidine, for example, reacts with dibenzoyl peroxide to form radicals. The radicals formed can initiate the radical polymerization of the methyl methacrylate. As the polymerization of the methyl methacrylate progresses, the viscosity of the cement dough can increase until it solidifies.

Polymethyl methacrylate bone cements can be mixed in suitable mixing beakers with the aid of spatulas by mixing the cement powder with the monomer liquid. Air bubbles may be incorporated in the bone cement dough, which can negatively influence the mechanical properties of the hardened bone cement. Other disadvantages are the need to measure the quantities manually and adequate mixing, which is not always ensured.

To avoid air inclusions in the bone cement dough, vacuum cementing systems are known, for example from U.S. Pat. Nos. 6,033,105 A, 5,624,184 A or 4,671,263 A. There is a need here to apply a vacuum. A further development in cementing technology is represented by cementing systems in which both the cement powder and the monomer liquid are already packaged in separate compartments and are mixed with one another directly before cement application in the cementing system. Such closed fully prepacked mixing systems are described in the documents EP0380867B1, EP0796653B1, or EP0692229B1.

Mixing devices in which an external device for squeezing out the bone cement is used are disclosed in WO9416951A1, EP1741413B1, EP3054880B1 and DE19718648A1. Mixing devices in which a vacuum is applied to enable the transport or mixing of a component or of the bone cement are known from DE102009031178B3, U.S. Pat. No. 8,662,736B2 and EP3093067B1. EP2393456B1 describes a mixing device in which the liquid is pressed into the powder by means of an overpressure.

### SUMMARY

It is the object of the invention to provide a particularly simple and cost-effective device for mixing the components for producing bone cement and for dispensing the bone cement produced. In particular, the object of the invention is to overcome, at least partially, the disadvantages known from the prior art.

The object is achieved by the device for mixing bone cement according to claim 1. Advantageous embodiments are specified in the dependent claims.

To achieve the object, a device for mixing bone cement is used. This comprises a first hollow body which encloses a first cavity for receiving a powder and has a first opening. The device further comprises a second hollow body which encloses a second cavity for receiving an ampoule and has a second opening. The first hollow body and the second hollow body are connectable to one another in such a way that the first opening and the second opening are fluidically connected to one another. The first hollow body is elastic so that a content of the first hollow body can be mixed from the outside by kneading.

By kneading, the components can be mixed particularly easily from the outside and without additional devices. A mixing rod or the like is not required, whereby the technical effort and the susceptibility to errors are significantly reduced. The device is very robust. In the simplest case, only two parts are required, which can also be produced cost-effectively. Handling is very simple and less susceptible to errors.

The elasticity of the first hollow body typically makes it possible to compress the first hollow body so that, for example, gas located therein, such as air, can be pressed out. In this way, the volume of the first cavity changes during the mixing and particularly good mixing can be achieved. For example, gas present in the first cavity can be temporarily or permanently pressed into the second cavity.

A hollow body is a body with a wall that encloses at least one cavity. The cavity can be open or closed. The first hollow body can be rubber-elastic and/or can be made of rubber. The first hollow body can also be referred to as an ampoule holder.

The second opening is permeable at least to liquids. In particular, the second opening is permeable to gases and liquids. The term “gas” refers in the present application only to the aggregate state and also includes gas mixtures, such as air. The second opening is dimensioned such that it is impossible for the ampoule to pass through the second opening. The first opening is in particular designed such that a powder can be introduced into the first cavity through the opening and/or such that a produced mixture, in particular a bone cement, can emerge from the first cavity through the opening.

The fluidic connection of the first opening to the second opening means such a connection that liquid or gas can flow from the surrounding environment from the first opening to the second opening and in particular also flow back.

Kneading from outside typically serves to mix the liquid component and the powder component to produce the bone cement. In particular, manual kneading is meant. During kneading, for example, one or more regions of the first hollow body can be compressed and released alternately. It is also possible to exert pressure progressively at different positions in the first hollow body; in particular, the first hollow body is elastic such that a temporary reduction of the outer diameter of the first hollow body by 50%, in particular by 70% relative to its unloaded extent is possible. Due to the elasticity, the first hollow body at least essentially assumes its original extent after application of external force has ceased.

The first hollow body can be designed such that a manual pressing out of the bone cement produced is possible by applying a manual force from the outside. In this way, it is possible to dispense the bone cement without an additional device for applying a force or generating a pressure or negative pressure.

In one embodiment, the second hollow body further comprises a third opening through which a gas can flow out of the second hollow body. If the first hollow body contains a gas, the gas can be pressed out of the first hollow body through the second hollow body and the third opening and in this way out of the device. This can typically be effected by applying a manual compressive force to the first hollow body. When the external force ceases, a negative pressure can be generated in the first hollow body, which in the meantime sucks the liquid, in particular the liquid leaving the ampoule, from the second cavity into the first cavity. In comparison to a pressure on the liquid, it is ensured in the case of bringing to the destination by suction that the liquid arrives only at the desired destination. Even in the event of a defect, the liquid thus cannot be pressed at another location.

For suction, it is not necessary to apply an external vacuum. The outlay in terms of equipment during use can thus be minimized and the application is thus independent of the presence of an external vacuum pump. No pump piston iis required to convey the monomer liquid to the cement powder.

The third opening is in particular arranged at an end facing away from the second opening, preferably at an end opposite the second opening. When the gas is being pushed out the device can thus be held in such a way that the third opening points upward. The gas located in the first cavity can thus be pressed out completely. A maximum negative pressure can thus be generated and a maximum amount of the liquid can flow into the first cavity.

The third opening can be closed, for example, with a gas-permeable closure, for example a gas-permeable cap. In this way, a penetration of contaminations is prevented. The third opening is in particular dimensioned such that it is impossible for the ampoule to pass through the third opening.

In particular, the third opening is configured such that a gas flow in both directions is possible. Sterilization with gas such as ethylene dioxide is easily possible in this way. In particular, sterilization can take place in the connected state of the two hollow bodies. In this case, the gas can also reach and penetrate the powder and sterilize the powder.

Preferably, the first hollow body is configured to apply after deformation a restoring force which counteracts the deformation. In this way, kneading for mixing is facilitated in comparison with, for example, a flexible bag, since the first hollow body only has to be compressed and the outwardly directed movement is automatically effected by the restoring force.

Preferably, a lower force is necessary for an elastic deformation of the first hollow body than for an elastic deformation of the second hollow body, provided such a force is possible. In this way, it is ensured that, in the connected state of the two hollow bodies, liquid is sucked out of the second hollow body into the first hollow body when a negative pressure is produced by pressing together the first hollow body. The second hollow body is prevented from being deformed, which would prevent the suction of the liquid into the first hollow body. For example, this can be achieved by a smaller wall thickness of the first hollow body compared to the second hollow body and/or by a softer or more flexible material of the first hollow body.

In one embodiment, the second hollow body is elastic at least in regions, such that the second hollow body can be bent from the outside in order to break open an ampoule located in the second cavity.

In particular, the second ampoule is broken open at a predetermined breaking point thereof. Such ampoules are usually produced from glass and/or have an ampoule body which is connected in one piece to a head. The head can be detached from the body by breaking, so that the substance contained in the ampoule, in particular liquid, is released. In particular, manual snapping is meant. Preferably, a shaped element is provided on the second hollow body in the region of the predetermined breaking point, on which shaped element the position of the predetermined breaking point can be seen from the outside.

In particular, a wall of the second hollow body in the region of the predetermined breaking point is particularly bendable in comparison to other points of the wall of the second hollow body. This can be achieved, for example, in a simple manner by a locally reduced wall thickness. In other words, the second hollow body in this case has a predetermined snapping point, at which a snapping is implemented when a force, in particular a manual force, is applied from the outside. In this way, a particularly simple and almost error-free opening of the ampoule can be made possible.

In one embodiment, the device is designed such that the head of the ampoule points in the direction of the second opening. The liquid thus does not have to flow past the body of the ampoule in order to flow out of the second opening.

In one embodiment, the second hollow body has a filter element. The filter element is arranged such that a flow flowing through the second opening is filtered.

A filter element is an element that enables a filtering of a medium in order to remove particles from the medium. The filter element can be designed, for example, as a sieve or as a packed bed of a material, in particular a porous material.

The flow through the second opening may be, for example, a gas flow, a flow of a liquid, a flow of a solid, for example of a powder, or any mixture of the examples mentioned. The filter element can prevent, for example, powder from entering the second cavity, glass splinters from escaping from the second cavity, and/or the bone cement produced from entering the second cavity.

In particular, the filter element is arranged in the region of the outer end of the second hollow body. For example, the filter element closes the second opening essentially flush. A few millimeters of deviation can be tolerated in this case. Penetration of substances into the second hollow body can thus be completely prevented. In addition, the filter element is particularly easy to install.

In one embodiment, the device comprises an ampoule received or receivable in the second cavity. Typically, the volume of the first cavity essentially corresponds to the volume of the ampoule. In other words, the cavity is at least essentially completely filled when the ampoule is arranged in the cavity. Typically, an inner contour of the second cavity corresponds at least essentially to an outer contour of the ampoule. In particular, the ampoule contains a liquid, for example for the production of bone cement. The ampoule can contain, for example, a monomer.

In one embodiment, the device comprises the powder received or receivable in the first cavity. In particular, it is powder for producing bone cement, preferably polymethyl methacrylate bone cement powder. In particular, the first hollow body is only partially filled with the powder. This facilitates mixing since a better mobility of the components to be mixed and of the mixture is achieved. For example, a volumetric proportion of more than 30%, preferably more than 40%, and in one embodiment approximately 50% or more and/or of less than 75%, preferably less than 60%, of the first cavity is filled with the powder. In particular, the remaining volume of the first cavity is filled with a gas.

If the device contains not only the ampoule with the liquid but also the powder, in other words is filled ready for use, the device can also be referred to as fully prepacked mixing system. It is therefore immediately ready for use for the production of bone cement. The device serves in particular for storing the two components and for mixing and discharging bone cement, in particular polymethyl methacrylate bone cement.

In one embodiment, the first cavity has a volume which corresponds at least to the sum of a volume of the powder and a volume of a liquid received or receivable in the ampoule. In this way, a sufficient volume is available for achieving a good movement of the mixture present in the first cavity and thus a good mixing of the components. A good quality of the bone cement can thus be achieved.

Preferably, the volume of the first cavity corresponds to at least the sum of the volume of the powder and twice the volume of the liquid receivable in the ampoule. In this way, the mixing is further simplified and at the same time a particularly high quality of the bone cement can be ensured. In one embodiment, the volume of the first cavity corresponds to at least the sum of the volume of the powder and the volume of the second cavity.

In one embodiment, the second cavity has a body region for receiving a body of the ampoule and/or a head region for receiving a head of an ampoule. In particular, an inner contour of the body region corresponds at least substantially to an outer contour of the body of the ampoule. The inner contour of the body region can, for example, have a circular cylindrical cross-section. In particular, an inner contour of the head region corresponds at least substantially to an outer contour of the head of the ampoule. Between the body region and the head region, a transition region can be provided in which edges and/or transitions can be of a rounded design. The body region and the head region are connected to one another so that the closed, filled ampoule can be accommodated in the second cavity.

In one embodiment, at least one first fluidic connection exists between an end of the body region facing the head region and the second opening. In this way, liquid from the body of the ampoule can flow from and/or be sucked out of the second hollow body even if the head region and a second fluidic connection possibly present there are blocked or clogged, for example by the broken-off head of the ampoule and/or by glass splinters. Typically, the amount of powder is dimensioned such that only the amount of liquid in the intended use position above the connection of the second fluidic connection is required for the production of the bone cement.

In one embodiment, a second fluidic connection exists between an end of the head region facing away from the body region and the second opening. In this way, liquid from the ampoule can flow out of and/or can be sucked out of the second hollow body. For example, a first flow duct which forms the first fluidic connection extends between an end of the head region facing away from the body region and the second opening.

For example, at least one second flow duct, which forms the second fluidic connection, extends between an end of the body region facing the head region and the first flow duct. In this case, for example, a lower region of the flow duct, which is connected to the second opening, serves as the first and second fluidic connection. A branch can be connected in which the first flow duct to the body part is diverted from the second flow duct to the head part, or vice versa. In other words, the first fluidic connection and the second fluidic connection can partially correspond. In one embodiment, two second fluidic connections are provided. These are, in particular, identical and/or are designed as second flow ducts as described.

In one embodiment, the second hollow body surrounds two second cavities, wherein each second cavity is configured for receiving an ampoule. In particular, a common second opening is provided, through which liquid can flow out of the two ampoules from the second hollow body. Typically, each second cavity has a separate opening, to which a respective flow duct is connected. The flow ducts can merge in particular in the interior of the second hollow body to form a single flow duct, which is connected to the common second opening. Alternatively, two second hollow bodies, in particular connected to one another, are present, each enclosing a cavity for receiving an ampoule.

The use of two ampoules instead of one larger ampoule makes it possible for the liquid volume in the ampoule to be less than 30 ml. This results in advantages due to restrictions relating to larger amounts according to hazardous substance regulations.

In one embodiment, a common snap line is provided for both second cavities, so that both ampoules located in the second hollow body can be broken open from the outside by a single snap action. A single snap action means a single snapping movement, which can be carried out in particular with a single force applied from the outside. Accordingly, only one movement is necessary in order to open both ampoules.

In particular, both ampoules can be broken open simultaneously. Typically, the second cavities are arranged parallel to one another and have the same orientation. The snap line then runs in particular perpendicularly to the longitudinal axis of the second cavities in line with a transition between a head region and a body region.

In one embodiment, the first hollow body has a first connecting element and/or the second hollow body has a second connecting element. In one embodiment, the first connecting element and the second connecting element are configured such that the first hollow body and the second hollow body are connectable to one another manually and releasably.

The first connecting element and the second connecting element are in particular directly connected to one another. The first connecting element and the second connecting element are typically designed as corresponding elements, for example as corresponding threads. The connection means a mechanical connection such that the two hollow bodies are fixedly coupled to one another and in this way can be moved, for example, together.

In particular, the connecting elements are designed such that, in the connected state, a longitudinal axis of the first hollow body and a longitudinal axis of the second hollow body lie one above the other. The liquid can thus flow into the powder via a straight, short flow path.

In one embodiment, the device further comprises an adapter which is connectable to the first connecting element. The adapter is preferably configured such that a mixture for producing bone cement is held back in the first hollow body.

In other words, with the adapter connected, the mixture is prevented from flowing out of the first hollow body. In this way, the contents of the first hollow body can be mixed when the adapter is connected. This facilitates mixing since the device in which the adapter is arranged instead of the second hollow body on the first hollow body is smaller and more manageable.

In particular, the adapter is configured such that gas can continue to escape from the first hollow body. Mixing is thus further improved, since more effective kneading becomes possible. In particular, the adapter comprises a filter element which holds the mixture back and/or allows gas to pass through. The filter element can be designed in the same way as the filter element of the second hollow body.

In one embodiment, the device further comprises a discharge element with a third connecting element. In particular, the discharge element can be connected to the first connecting element of the first hollow body.

The discharge element serves to discharge the produced bone cement from the first hollow body. In particular, this can be achieved by pressing together the first hollow body. Instead of the second connecting element, the third connecting element is typically connected to the first connecting element after the release of the second connecting element from the first connecting element. Connection means a mechanical coupling such that a fluidic connection exists between the first cavity and an interior of the discharge element. In particular, the discharge element can be connected manually to the first connecting element. In particular, a mechanically interlocking and/or frictionally interlocking connection is meant. In this way, particularly simple handling is made possible.

The discharge element preferably has a nozzle through which the bone cement can be conveyed precisely to a target position. The nozzle is preferably arranged at the end of the discharge element opposite the third connecting element.

In one embodiment, the first hollow body is produced from a material having a Shore A hardness between 30 and 80.

In one embodiment, the first hollow body is produced from a material having a Shore A hardness greater than 30, in particular greater than or equal to 40, and preferably greater than or equal to 50 and/or less than 80, preferably less than 70, in particular less than 60. Such materials have proven to be particularly advantageous with regard to the required elasticity.

A further aspect of the invention is a method for producing bone cement, in particular using the device according to the invention. The method comprises kneading an elastic first hollow body from the outside, in particular manually kneading, for mixing a content of the first hollow body. In particular, the first hollow body encloses a first cavity for receiving a powder and has a first opening. In particular, a second hollow body is present. In particular, the second hollow body encloses a second cavity for receiving an ampoule and has a second opening. In particular, during the kneading, the first opening and the second opening are fluidically connected to one another. In particular, the first hollow body is positioned such that the first opening points upward.

In one embodiment, the method comprises applying a force to the first hollow body from the outside. In this way, a gas present in the first hollow body can flow out of the first hollow body through the first opening. In particular, the gas can flow through the second opening into the second hollow body, can flow past an ampoule present in the second cavity and/or can leave the second cavity through a third opening of the second hollow body. In one embodiment, the method comprises a snapping of the second hollow body from the outside, so that the ampoule is broken open and a liquid present in the ampoule is released. In one embodiment, the method comprises releasing the force applied to the first hollow body. In this way, a negative pressure can be generated by a restoring force of a wall of the first hollow body in order to suck the released liquid into the first hollow body. In particular, the kneading then follows. In one embodiment, the method comprises removing the second hollow body from the first hollow body, connecting a discharge element to the first hollow body and/or discharging the bone cement produced by applying an external force to the first hollow body.

In one embodiment, the force with which the gas is pressed out of the first hollow body is applied before the ampoule is broken open. In an alternative embodiment, the ampoule is first broken open and then a single or multiple application of a force on the first hollow body is provided from the outside. In this way, the gas can flow out in one or more parts and a corresponding portion of the liquid can be sucked in by the resulting negative pressure.

Exemplary embodiments of the invention are also explained in greater detail below with reference to figures. Features of the exemplary embodiments can be combined individually or in a plurality of the claimed subjects, unless otherwise indicated. The claimed scope of protection is not limited to the exemplary embodiments.

### BRIEF DESCRIPTION

In the drawings:

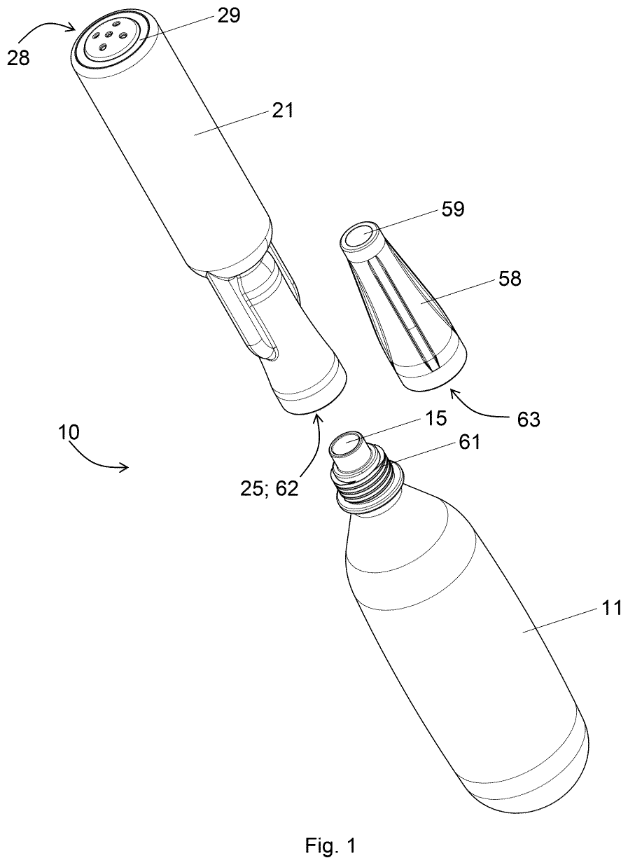

FIG. 1: shows a perspective view of a device according to the invention,

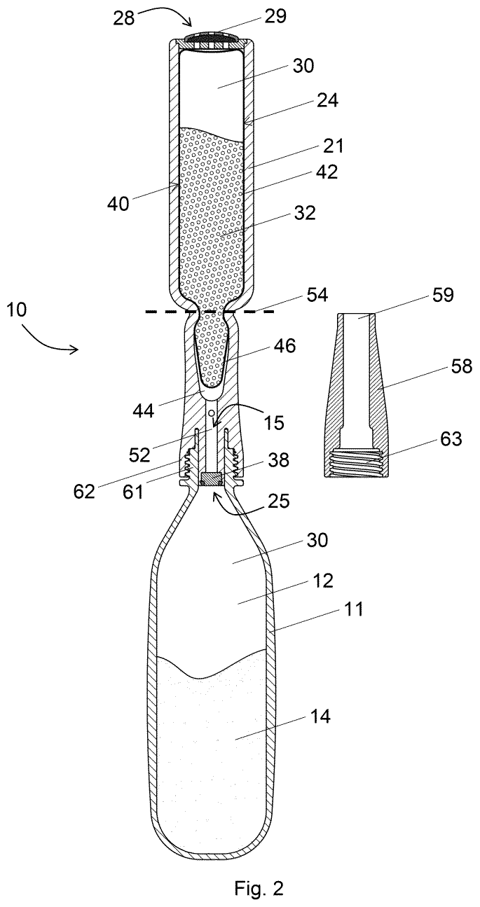

FIG. 2: shows a sectional view of a device according to the invention,

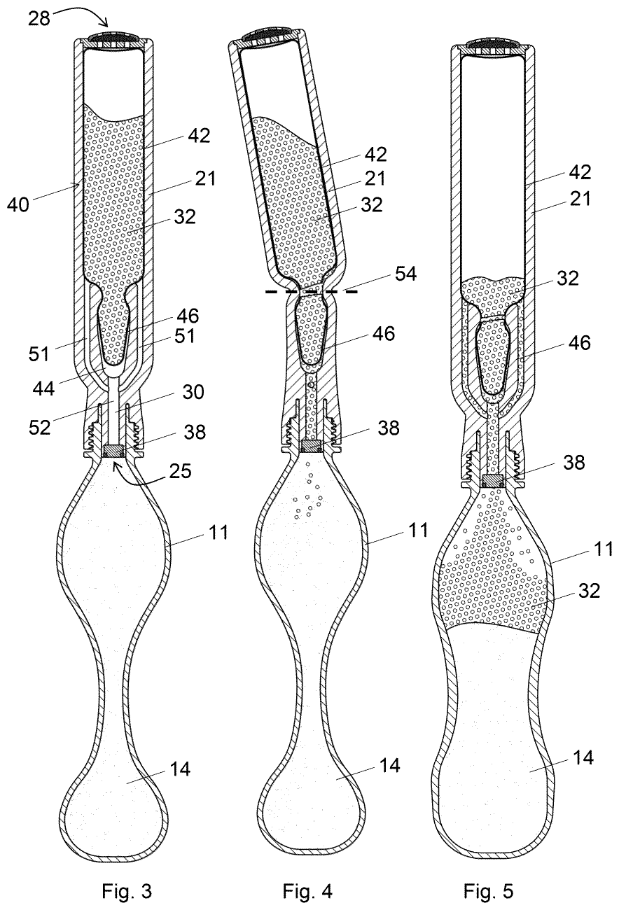

FIG. 3: shows a further sectional view of a device according to the invention,

FIG. 4: shows a further sectional view of a device according to the invention,

FIG. 5 shows a further sectional view of a device according to the invention,

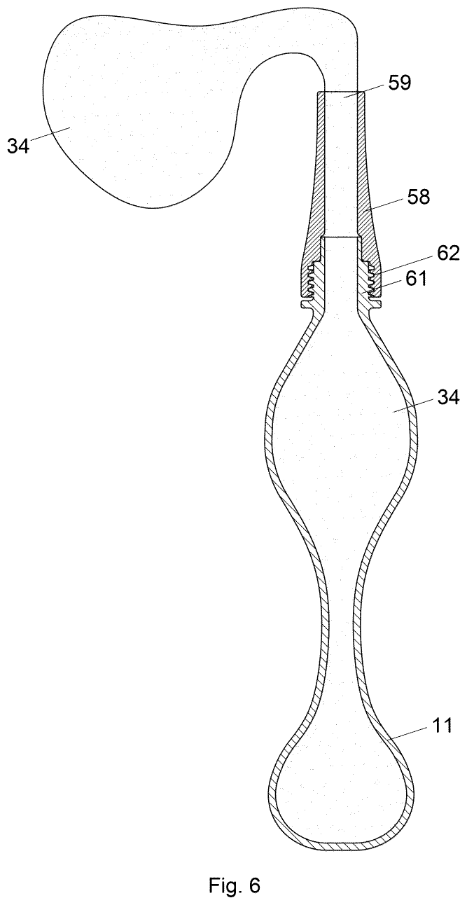

FIG. 6: shows a further sectional view of a device according to the invention,

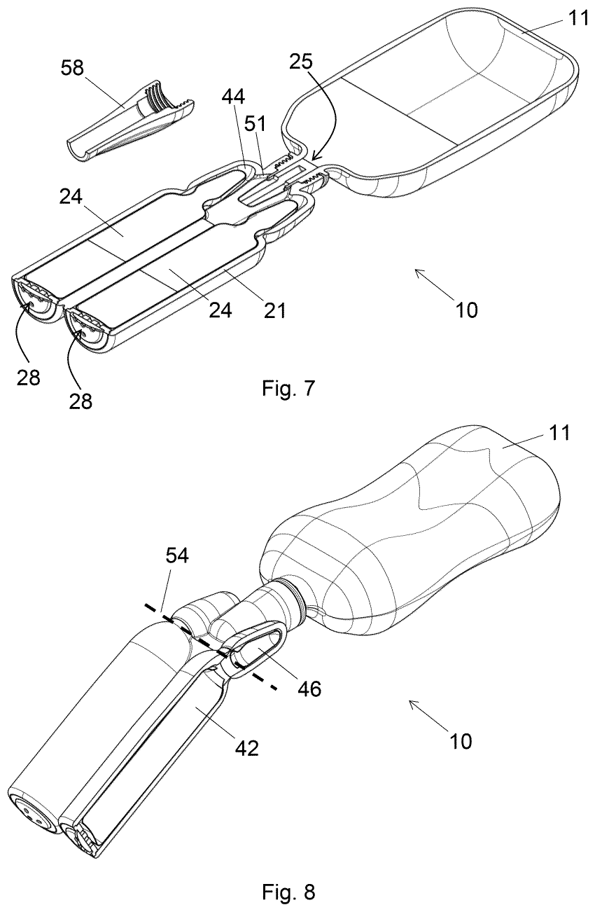

FIG. 7 shows a sectional perspective view of a device according to the invention,

FIG. 8: shows a perspective view of a device according to the invention,

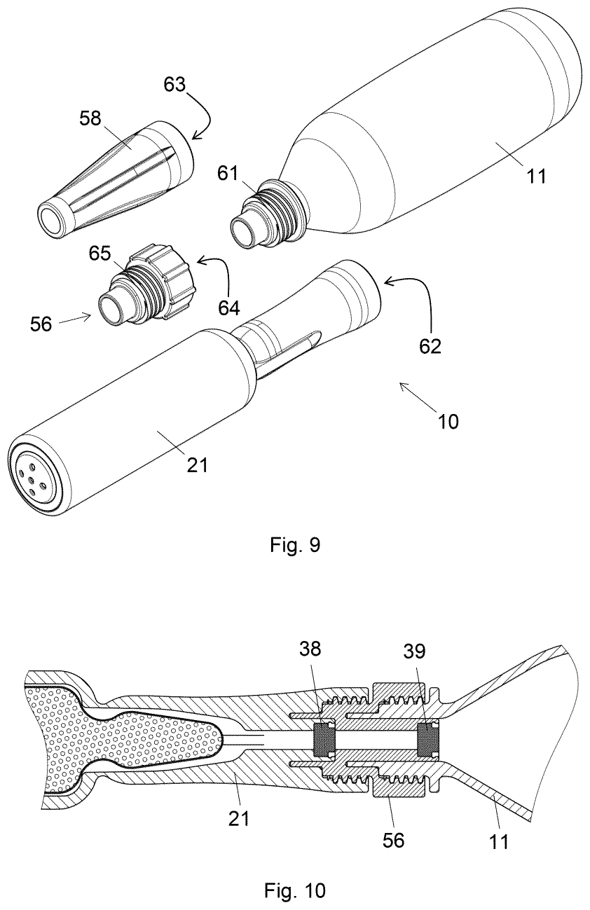

FIG. 9: shows a perspective view of a device according to the invention, and

FIG. 10 shows a sectional detail drawing of a device according to the invention.

### DETAILED DESCRIPTION

FIG. 1 shows a device 10 for mixing bone cement. The device 10 comprises a first hollow body 11, a second hollow body 21 and an optional discharge element 58. In particular, each of the hollow bodies 11, 21 is made of rubber and/or plastic. The first hollow body 11 comprises a first opening 15. A first connecting element 61 in the form of an external thread is arranged in the region of the first opening 15. The second hollow body 21 comprises a second opening 25 and a third opening 28. The third opening 28 is closed with a gas-permeable cap 29. The cap 21 can be designed to be removable in order to allow an ampoule to be inserted into the second hollow body 21. The two openings 25, 28 are arranged in particular centrally at opposite ends of the second hollow body 21.

A second connecting element 62 in the form of an internal thread is located in the region of the second opening 25. The first hollow body 11 and the second hollow body 21 can be mechanically connected to one another by means of the connecting elements 61, 62. In the connected state of the two hollow bodies 11, 21, the first opening 15 and the second opening 25 are fluidically connected to one another.

The discharge element 58 comprises a nozzle 59 for discharging bone cement and a third connecting element 63 in the form of an internal thread, which in particular corresponds to the internal thread of the second connecting element 62. In this way, the discharge element 58 can be mechanically connected to the first connecting element 61 of the first hollow body 11 instead of the second hollow body 21.

FIG. 2 shows the device 10, which can correspond to the device of FIG. 1, in a sectional view. As described above, the first hollow body 11 and the second hollow body 21 are mechanically connected to one another by means of the connecting elements 61, 62. The first hollow body 11 encloses a first cavity 12, which is partially filled with powder 14 and partially with a gas 30, for example air. The second hollow body 21 encloses a second cavity 22, in which an ampoule 24 is present. The ampoule 24 is partially filled with a liquid 32 and partially filled with a gas 30 or completely filled with the liquid 32.

The ampoule 24 is composed of an essentially circular-cylindrical body 42 and a head 46 adjoining the same. In FIG. 2, the head 46 points downward. At the transition between the body 42 and the head 46 there is a predetermined breaking point in order to separate the head 46 from the body and in this way release the liquid 32. The second cavity 22 of the second hollow body 21 is accordingly divided into an essentially circular-cylindrical body region 40 for receiving the body 42 and a head region 44 adjoining the same for receiving the head 46. The second hollow body 21 is elastic at least in the region of the predetermined breaking point, so that the ampoule 24 can be opened as described by snapping from the outside along the snap line 54.

A second fluidic connection 52 in the form of a straight flow duct is located between the lower end of the head region 44 facing away from the body region 40 and the second opening 25. At the lower end of the flow duct in the region of the second opening 25, there is a filter element 38 with which a flow flowing through the second opening 25 can be filtered. In this way, for example, glass shards which are created when the ampoule 24 is opened can be held back when the liquid 32 is flowing through the second opening 25.

FIGS. 3 to 5 show successive steps of a method for producing a powder cement. The device 10 used for this purpose corresponds in particular to the device from FIG. 2. In FIGS. 3 and 5, the device 10 is shown rotated by 90° along its longitudinal axis. It is shown here that, in addition to the second fluidic connection 52, two first fluidic connections 51 also exist between the second cavity 22 and the second opening 25. Two, for example curved, flow ducts run between the lower end of the body region 40 facing the head region 44 and the straight flow duct of the second fluidic connection 52. The first fluidic connections 51 are therefore located above or at least at the same height as the predetermined breaking point. In this way, the liquid 32 can flow through the second opening 25 even if the head region 44 is clogged by the broken-off head 46 of the ampoule 24 or by glass shards.

FIG. 3 shows that a manual force is being applied from the outside to the first hollow body 11. The first hollow body 11 is pressed together. Gas present above the powder 14 in this way flows through the second opening 25 and the fluidic connections 51, 52 into the second cavity 22, past the ampoule 24 and out of the second hollow body 21 through the third opening 28. The filter element 38 can prevent powder 14 from getting into the second hollow body 21.

FIG. 4 shows the snapping of the second hollow body 21 along the snap line 54 by the application of manual force from the outside. As a result, the head 46 of the ampoule 24 is separated from the body 42 and the liquid 32 is released. The liquid 32 begins to flow downwards. The manual force applied to the first hollow body 11 is now removed. Due to the restoring force exerted by the wall of the first hollow body 11, a negative pressure is formed in the first hollow body 11 and sucks the liquid 32 downwards in addition to the action of gravity. FIG. 5 shows the state after a large part of the liquid 32 has already flowed into the first hollow body 11. As soon as the first hollow body 11 has assumed its original shape again and/or the required amount of liquid 32 has flowed into the first hollow body 11, the elastic first hollow body 11 can be kneaded from the outside in order in this way to mix the powder 14 with the liquid 32 as homogeneously as possible. The filter element 38 here prevents the mixture from flowing into the second hollow body 21. In this way, a processed bone cement is produced in the first cavity 12.

FIG. 6 shows the dispensing of the bone cement that has been produced. Instead of the second hollow body, the discharge element 58 is now fastened to the first connecting element 61 of the first hollow body 11. By applying a manual pressure force from the outside to the flexible first hollow body 11, the produced bone cement 34 is pressed out through the nozzle 59.

FIGS. 7 and 8 show another embodiment of the device 10 according to the invention. Deviating from the previously described devices, the second hollow body 21 here surrounds two second cavities 22. An ampoule 24 is present in each second cavity 22. Each of the second cavities 22 is connected to the second opening 25 by means of one or more fluidic connections. FIG. 8 shows that a common snap line 54 for the two second cavities 22 is provided. The liquid from the two ampoules 24 can thus be released from the outside by a single snapping movement.

FIGS. 9 and 10 show another embodiment of the device 10 according to the invention. In addition to what has already been described, the device 10 shown here comprises an adapter 56. This comprises a fourth connecting element 64 in the form of an internal thread which corresponds essentially to the second connecting element 62. The adapter 56 further comprises a fifth connecting element 65 in the form of an external thread which corresponds essentially to the first connecting element 61. The adapter 56 can thus be screwed onto the first connecting element 61 and onto the second connecting element 62 and can be arranged accordingly between the first hollow body 11 and the second hollow body 21, as shown in FIG. 10. The liquid from the ampoule can thus flow through the adapter 56 into the first cavity 12.

The adapter 56 further comprises a second filter element 39. This is designed in particular like the first filter element 38. The second filter element 39 is located in particular at the end of the flow duct, extending through the adapter 56, which is facing the first hollow body 11 during intended use. In this way, the second filter element 39 prevents the mixture for producing bone cement from coming out of the first hollow body 11 when the latter is being kneaded. It is thus possible to carry out the kneading from the outside for mixing the bone cement, while the second hollow body 21 is separated from the first hollow body 11 and only the adapter 56 is arranged on the first hollow body 11. In this way, the device is more manageable, which facilitates mixing. Overall, the first hollow body 11 and the second hollow body can thus be connected directly or indirectly with an interposed adapter.

### LIST OF REFERENCE NUMERALS

Device 10
First hollow body 11
First cavity 12
Powder 14
First opening 15
Second hollow body 21
Second cavity 22
Ampoule 24
Second opening 25
Third opening 28
Cap 29
Gas 30
Liquid 32
Bone cement 34
Filter element 38
Second filter element 39
Body region 40
Body 42
Head region 44
Head 46
First fluidic connection 51
Second fluidic connection 52
Snap line 54
Adapter 56
Discharge element 58
Nozzle 59
First connecting element 61
Second connecting element 62
Third connecting element 63
Fourth connecting element 64
Fifth connecting element 65

1. A device for mixing bone cement, comprising a first hollow body which encloses a first cavity for receiving a powder and has a first opening, and a second hollow body which encloses a second cavity for receiving an ampoule and has a second opening, wherein the first hollow body and the second hollow body are connectable to one another in such a way that the first opening and the second opening are fluidically connected to one another, wherein the first hollow body is elastic, such that the content of the first hollow body can be mixed from the outside by kneading.

2. The device according to claim 1, wherein the second hollow body further comprises a third opening through which a gas can flow out of the second hollow body.

3. The device according to claim 1, wherein the second hollow body is elastic at least in regions, such that the second hollow body can be bent from the outside in order to break open an ampoule present in the second cavity.

4. The device according to claim 1, wherein the second hollow body has a filter element, the filter element being arranged such that a flow flowing through the second opening is filtered.

5. The device according to claim 1, wherein the device comprises an ampoule received in the second cavity.

6. The device according to claim 1, wherein the device comprises a powder received in the first cavity.

7. The device according to claim 1, wherein the first cavity has a volume which corresponds at least to the sum of a volume of the powder and a volume of a liquid receivable in the ampoule.

8. The device according to claim 1, wherein the second cavity has a body region for receiving a body of the ampoule and a head region for receiving a head of an ampoule, at least one first fluidic connection existing between an end of the body region facing the head region and the second opening.

9. The device according to claim 1, wherein a second fluidic connection exists between an end of the head region facing away from the body region and the second opening.

10. The device according to claim 1, wherein the second hollow body encloses two second cavities, each second cavity being configured to receive an ampoule.

11. The device according to claim 3, wherein a common snap line is provided for the two second cavities, so that the two ampoules present in the second hollow body can be broken open from the outside by a single snap action.

12. The device according to claim 1, wherein the first hollow body has a first connecting element and the second hollow body has a second connecting element, the first connecting element and the second connecting element being configured such that the first hollow body and the second hollow body are connectable to one another manually and releasably.

13. The device according to claim 1, wherein the device further comprises an adapter which is connectable to the first connecting element and is configured such that a mixture for producing bone cement is held back in the first hollow body.

14. The device according to claim 12, wherein the device further comprises a discharge element having a third connecting element which can be connected to the first connecting element of the first hollow body.

15. The device according to claim 14, wherein the first hollow body is made of a material having a Shore A hardness between 30 and 80.
